(19) United States
(12) Patent Application Publication
(10) Pub. No.: US 2025/0319268 A1
(43) Pub. Date: Oct. 16, 2025

(54) FLUID SCAVENGING SYSTEM AND PROCESS FOR CONVEYING A FLUID

(71) Applicant: Drägerwerk AG & Co. KGaA, Lübeck (DE)

(72) Inventors: Ralf HEESCH, Lübeck (DE); Robert SCHMID, Lübeck (DE)

(21) Appl. No.: 19/174,099

(22) Filed: Apr. 9, 2025

(30) Foreign Application Priority Data
Apr. 12, 2024 (DE) 10 2024 110 226.8

(51) Int. Cl. A61M 16/00; A61M 16/10; A61M 16/12

(52) U.S. Cl. CPC A61M 16/009; A61M 16/0009; A61M 16/024; A61M 16/104; A61M 16/12; A61M 2205/15

(57) ABSTRACT

A scavenging system and a process have a conveying state with a suction arrangement (200) conveying a fluid from a source to a sink. In a rest state, the conveyance of fluid is prevented or interrupted. A plug (15) can be plugged into a socket (16), and, if plugged in, a fluid connection is established between the source and the sink. In a conveying switching position, a switching element (38.1, 38.2) sets the scavenging system to the conveying state, and in a rest switching position to the rest state. A passive resetting element (39.1, 39.2) holds the switching element in the conveying switching position in a rest state. A switched-on actuator (42.1, 42.2) switches the switching element to the rest switching position against a resetting force of the resetting element. A signal-processing control unit detects an event that no fluid is escaping from the source and switches the actuator on.

### CROSS REFERENCE TO RELATED APPLICATIONS

This application claims the benefit of priority under 35 U.S.C. § 119 of German Application 10 2024 110 226.8, filed Apr. 12, 2024, the entire contents of which are incorporated herein by reference.

### TECHNICAL FIELD

The present invention relates to a scavenging system for a fluid. In particular, the fluid is a gas mixture with an anesthetic gas. Furthermore, the invention relates to a process for conveying a fluid from a source to a sink.

In one application, the source is a ventilator for artificial ventilation (also known as artificial respiration) of a patient, wherein the patient is supplied with a gas mixture comprising oxygen and at least one anesthetic, optionally a medication. The sink is a receiving (absorption) system for excess gas mixture, wherein the received gas mixture generally comprises anesthetic.

### SUMMARY

It is an object of the invention to provide a fluid scavenging system and a process for conveying a fluid, which should have a higher operational reliability than known fluid scavenging systems and processes.

The object is attained by a fluid scavenging system with features according to the invention and by a process with features according to the invention. Advantageous embodiments are disclosed in the dependent claims and in the description, drawings and claims. Advantageous embodiments of the scavenging system according to the invention are, where appropriate, also advantageous embodiments of the process according to the invention and vice versa.

The scavenging system according to the invention is configured to convey a fluid. The fluid is preferably a gas or gas mixture, in particular a gas mixture which is exhaled by an artificially ventilated patient, and which may contain anesthetics and/or a medication.

The scavenging system according to the invention comprises a plug and a socket. If the plug is inserted into the socket, a fluid connection between a source and a sink is established or can be established. A fluid can flow through this fluid connection from the source to the sink. This fluid connection is interrupted at least if the plug is not inserted into the socket. The fluid connection can also be temporarily interrupted if the plug is inserted.

The scavenging system can be set to a conveying (delivery) state and a rest (idle) state. In the conveying state, the fluid connection is established. If the scavenging system is in the conveying state, a suction arrangement is able to convey fluid from the source to the sink. In particular, the suction arrangement sucks the fluid from the source and conveys the fluid to the sink. In the rest state, the fluid connection is interrupted and/or the suction arrangement is switched off. It is possible that in the rest state the plug is inserted into the socket and the suction arrangement is switched off.

A switching element of the scavenging system can be switched to a conveying switching position and a rest switching position. The switching element in the conveying switching position sets the scavenging system to the conveying state and holds (keeps) it in this state. The switching element in the rest switching position sets the scavenging system to the rest state and holds it in this state.

A resetting element of the scavenging system is implemented (configured) as a mechanical component and has a rest state. A “mechanical component” is understood to be a component that is able to execute a movement and does not need to be supplied with electrical, pneumatic, or hydraulic energy to do so. Preferably, the resetting element comprises at least one mechanical or pneumatic spring.

In the rest state, the resetting element holds the switching element in the conveying switching position. If the resetting element is deflected from the rest state, the resetting element exerts a resetting force, and the resetting force strives to return the resetting element to the rest state and hold it in the rest state. In particular, the resetting force is generated mechanically and/or pneumatically. The resetting element is connected to the switching element in such a way that the resetting force of the resetting element strives to switch the switching element to the conveying switching position and/or to hold (keep) it in this position.

An actuator of the scavenging system can be switched on and off by a corresponding control. The activated actuator is able to switch the switching element against the resetting force from the conveying switching position to the rest switching position and hold it there, thereby transferring the scavenging system to the rest state. If the actuator is switched off, the resetting element holds the switching element in the conveying switching position. Switching off the actuator causes the resetting element to move the switching element into the conveying switching position.

A signal-processing control unit is able to control the actuator and thereby switch it on and preferably also switch it off again. The control unit is able to monitor the scavenging system to determine whether at least one possible predetermined event has actually occurred. This possible event or each possible event means or results in no fluid escaping from the source.

The control unit is configured as follows: If the control unit has positively detected the or at least one such event, the control unit switches on the actuator. The fact that an event is “positively detected” means the following: The control unit has determined with sufficient certainty that this event has actually occurred. In case of doubt, the control unit does not switch on the actuator.

The process according to the invention is carried out using such a scavenging system. The process comprises the following steps:
The plug is plugged into the socket or is in a plugged-in state.
The switching element is in the or is switched to the conveying switching position.
The switching element in the conveying switching position moves or holds the scavenging system in the conveying state.

If the scavenging system is in the conveying state, the following steps are carried out:
The fluid connection between the source and the sink is or will be established. This fluid connection guides through the plug and through the socket.
The suction arrangement conveys the fluid through the fluid connection from the source to the sink, preferably by suction.
The actuator is switched off.
The resetting element is in the rest state and holds the switching element in the conveying switching position.
While being deflected from the rest state, the resetting element exerts a resetting force. The resetting force strives to switch the switching element to the conveying switching position and/or to hold the switching element in the conveying switching position.
The control unit monitors the scavenging system and/or the source for at least one predetermined possible event in which no fluid escapes from the source.

The execution of the further steps described below is triggered
while the plug is inserted into the socket and
if the control unit has detected that a specified possible event has actually occurred.

If this event is positively detected, it is clear that no fluid is escaping from the source and therefore no fluid needs to be conveyed to the sink.

In this situation, the following steps are then carried out:
The control unit switches the actuator on.
The activated actuator switches the switching element to the rest switching position, against the resetting force of the resetting element, and holds it in this position.
In the rest switching position, the switching element causes the scavenging system to be set to the rest state and held in the rest state. In the rest state of the scavenging system, the conveyance of fluid through the fluid connection is interrupted and/or prevented.

While the scavenging system is in the conveying state, the scavenging system conveys fluid from the source to the sink, thereby preventing too much fluid from accumulating in the source in many cases and, for example, preventing the formation of an undesirable overpressure in the source.

In many cases, a safe state is present if the scavenging system is in the conveying state and conveys fluid from the source to the sink. The resetting element strives to keep the switching element in the conveying switching position and thereby to ensure that the scavenging system is and remains in the conveying state. Because the resetting element is configured as a mechanical component and, in its rest state, holds the switching element in the conveying switching position, the scavenging system remains in the conveying state even if an electrical or pneumatic or hydraulic supply to the switching element has failed or been interrupted or the switching element itself has failed or been switched off. The scavenging system remains in the conveying state even if the actuator or a supply to the actuator has failed. The invention thus increases the reliability that actually the safe state is established.

If, on the other hand, there is no excess fluid in the source, it is not necessary to convey fluid from the source to the sink. In this situation, the scavenging system can be in the rest state and then consumes less energy than in the conveying state. This configuration saves electrical energy compared to a state in which the scavenging system is permanently in the conveying state.

The invention accomplishes the following: If it has been positively determined that there is no excess fluid in the source and it is therefore a safe condition that the scavenging system is at rest, the actuator is switched on, thereby transferring the switching element to the rest switching state and holding it in this state. This step is performed against the resetting force of the resetting element. In case of doubt, the scavenging system is held in the conveying state or switched to the conveying state. This case of doubt exists if it cannot be determined with sufficient certainty that no excess fluid has occurred, nor can it be determined with sufficient certainty that excess fluid is present and must be discharged.

As already explained, the safe state is usually that the scavenging system is in the conveying state. In the conveying state, the actuator is usually switched off and does not consume any electrical energy and does not need to be supplied with a hydraulic or pneumatic fluid

Thanks to the invention, it is not necessary for the actuator to be able to move the switching element in both directions. Rather, it is sufficient for the actuator to be able to move the switching element into the rest switching position against the resetting force. If the actuator is switched off, the resetting element moves the switching element to the conveying switching position and holds it there. Because the conveying switching position of the actuator generally leads to a safe state, it is often not necessary for the actuator to be able to quickly transfer the switching element to the rest switching position. The actuator therefore only needs to apply enough force to overcome the resetting force of the resetting element.

According to the invention, at least one possible detectable event is predetermined (given) in which it is established (is for sure) that no fluid is escaping from the source. For example, it is established that no fluid is escaping from a device comprising the source. An indication of this can be that this device does not receive or consume any electrical current. The control unit is able to detect this or any predetermined event or to determine that no such event has occurred. Various embodiments are possible as to which such events are predetermined. It is also possible that at least one event is predetermined in which fluid is certain to escape or at least is possibly likely to escape from the source.

In one embodiment, the or a detectable event is the event that the source—or a device with the source—is switched off or has been switched off and has not been switched on again. As a rule, a switched-off source is not capable of expelling fluid. The control unit can detect this event.

In one embodiment, the control unit is able to detect an indication that fluid is actually escaping from the source. Optionally, the control unit can also detect an indication that the source is ready to expel fluid. If the control unit has detected such an indication, the control unit causes the actuator to be switched off or ensures that the actuator is switched off. This embodiment therefore causes the scavenging system to be transferred to a safe state, namely the conveying state, if fluid escapes from the source or the source is at least ready to expel fluid.

In one embodiment of this configuration, the control unit is able to detect one of the following three indicators as an indication that fluid is escaping from the source and/or that the source is ready for operation:
The source absorbs or consumes electrical energy, for example for an internal electrical consumer. The energy consumption is above a predefined lower threshold.
The source emits (outputs) electrical energy, for example to a connected external electrical consumer. The energy output is above a predefined lower threshold.
Fluid flows into the source.

In one implementation, the control unit is able to detect an indication for the following event: A data connection between the source or a device comprising the source on the one hand and the control unit on the other hand is interrupted. For example, the control unit sends a query to the device with the source, but the device does not respond. If the control unit has detected an indication of an interrupted data connection, the control unit causes the following: The step is triggered that the actuator is switched off, or it is ensured that the actuator is and remains switched off. The resetting element switches the switching element to the conveying switching position. This configuration establishes a safe state in the event of an interrupted data connection, namely that the scavenging system is in the conveying state.

According to the invention, the scavenging system can be set to a conveying state and to a rest state. In the conveying state, the suction arrangement conveys fluid from the source to the sink. Different configurations are possible as to how a rest state of the scavenging system is brought about.

In one embodiment, the suction arrangement can be switched on and off. If the suction arrangement is switched on, the scavenging system is in the conveying state; if the suction arrangement is switched off, the scavenging system is in the rest state. The switching element is able to set the suction arrangement to either a switched-on or a switched-off state and keep it in this state. The configuration in which the suction arrangement can be switched off saves electrical energy compared to a configuration in which the suction arrangement is permanently switched on. Preferably, the suction arrangement is assigned to a specific source.

In accordance with the invention, the plug of the scavenging system can be plugged into the socket. In the embodiment in which the suction arrangement can be switched on and off, the plug is preferably configured as follows: The resetting element and the actuator are components of the plug. Furthermore, the plug comprises an actuating element. The actuating element is mounted such that it can be moved relative to a housing of the plug. If the plug is inserted into the socket, the actuating element is also movable relative to the socket.

The actuating element can be moved into an actuating position. If the actuating element is in the actuating position, the actuating element actuates (operates) the switching element. The actuation causes the switching element to be transferred to the conveying switching position. The actuated switching element switches the suction arrangement on and/or holds the suction arrangement in the switched-on state. The actuated switching element therefore causes the suction arrangement to be switched on and the scavenging system to be in the conveying state.

Various configurations are possible as to how the actuating element is moved. It is possible that the actuating element can be controlled externally or actuated directly by a person. The following describes an implementation that does not require external control or actuation.

According to the invention, the resetting element has a rest state. If the resetting element is deflected from the rest state, the resetting element exerts a resetting force. In one embodiment, the resetting element in the rest state holds the actuating element just described in the actuating position. The resetting element in the rest state thereby ensures that the scavenging system remains in the conveying state and thus guarantees a safe state.

According to the invention, the control unit is able to switch on the actuator. In the embodiment just described, the switched-on actuator is able to move the actuating element out of the actuating position, namely against the resetting force of the resetting element. The switched-on actuator thereby causes
the switching element to be transferred to the rest switching position,
the suction arrangement to be switched off, and
this step transfers the scavenging system to the rest state and keeps it in the rest state.

According to the invention, if the plug is inserted into the socket, a fluid connection is or can be established between the source and the sink. In one embodiment, at least one switching valve is arranged in the fluid connection, with the fluid connection leading from the source to the sink. In this embodiment, the or each switching valve is part of the switching element. The or each switching valve can be switched to a conveying switching position and to a rest switching position. These two switching positions are the switching positions of the switching element according to the invention. In the conveying switching position, the switching valve is open and releases the fluid connection. In the rest switching position, the switching valve is closed and interrupts the fluid connection.

If the resetting element is in the rest state, it holds the switching valve in the conveying switching position. The activated actuator is able to switch the switching valve to the rest switching position, against the resetting force of the resetting element, and hold it in this position. If the actuator is switched off again, the resetting element returns the switching valve to the conveying switching position.

The configuration with the or at least one switching valve makes it possible for the suction arrangement to be permanently switched on. In many cases, a permanently switched-on suction arrangement is able to convey fluid from various sources, in particular to suck in fluid. In addition, the suction arrangement is immediately available if required and does not have to be started up first. It is possible to provide such a permanently switched-on suction arrangement with its own power supply unit, so that the suction arrangement is still ready for operation even if a stationary power supply network fails. The arrangement with the or at least one switching valve can also be combined with a suction arrangement that can be switched on and off.

In one embodiment, the source is a component of a device, preferably a medical device, in particular a ventilator for artificial ventilation. The fluid is expelled or escapes from this device. The control unit preferably comprises its own housing. The control unit is arranged at a distance (remote) from the device with the source. Preferably, the control unit comprises its own power supply unit and/or its own coupling element, which coupling element can be connected to a stationary power supply network. The fact that the control unit is spaced apart makes it easier to integrate the invention into an existing scavenging system. In many cases, the device with the source does not need to be modified, or modified only slightly. In addition, the control unit can be monitored and replaced more easily.

The invention also relates to a system which comprises a scavenging system according to the invention, a source for a fluid, and a sink for the fluid. If the plug of the scavenging system is plugged into the socket, a fluid connection between the source and the sink of the system is established or can be established. If the plug is not plugged in, this fluid connection is interrupted. The embodiments and advantages of the scavenging system just described also apply to this system.

In one embodiment, the system comprises a ventilator. The ventilator is configured to artificially ventilate (respirate) a patient and is capable of expelling a breathable gas mixture. While the ventilator performs the artificial ventilation, a patient-side coupling unit is arranged in and/or on the patient's body. The patient-side coupling unit comprises, for example, a breathing mask or a tube. During artificial ventilation, a fluid connection is established permanently or at least temporarily between the ventilator and the patient-side coupling unit. The breathable gas mixture expelled by the ventilator flows through this fluid connection to the patient-side coupling unit. The patient can inhale this gas mixture.

The scavenging system according to the invention is capable of conveying a fluid from the source to the sink. According to the embodiment just described, the source is located in the ventilator or in the fluid connection between the ventilator and the patient-side coupling unit. The fluid provided by the source preferably comprises gas exhaled by the artificially ventilated patient. The sink comprises a suction arrangement for the fluid. Thanks to the suction arrangement, the exhaled gas is prevented from escaping into the environment.

It is possible that the air that has been exhaled by the patient is fed into the environment. In a different implementation, however, a ventilation circuit is established between the ventilator and the patient-side coupling unit. This configuration prevents gas exhaled by the patient from entering the system's environment. This is particularly important in the following situation: The gas mixture, which is expelled from the ventilator and flows to the patient-side coupling unit, contains at least one anesthetic and optionally a medication. As a result, the patient is anaesthetized or at least sedated. The gas exhaled by the patient therefore contains an anesthetic. The ventilation circuit prevents this anesthetic from entering the system's environment. The source of the fluid is in the ventilation circuit. The fluid is, for example, excess gas from the ventilation circuit.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first embodiment of the ventilation arrangement according to the invention with a vacuum source that can be switched on and off;

FIG. 2 is a schematic view of a second embodiment of the ventilation arrangement according to the invention with a switching valve;

FIG. 3 is a schematic view showing the plug of the fluid scavenging system and a first configuration of the vacuum source that can be switched on and off;

FIG. 4 is a schematic view showing the plug of FIG. 3 and a second embodiment of the vacuum source; and

FIG. 5 is a schematic view showing the control unit that switches the actuator of the plug on and off.

### DESCRIPTION OF PREFERRED EMBODIMENTS

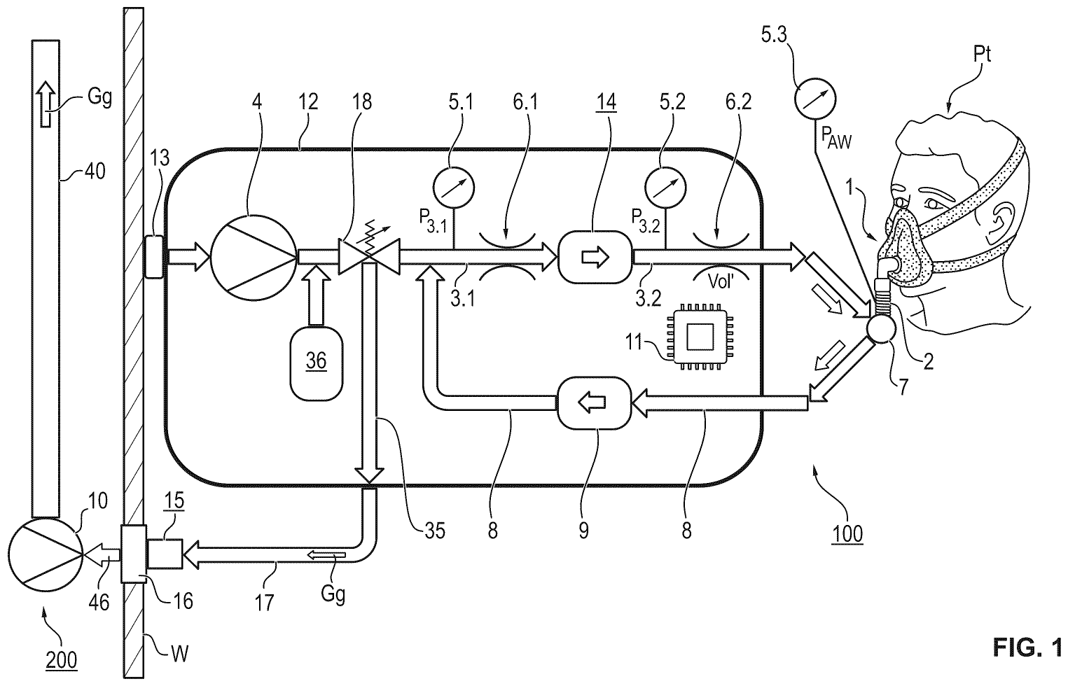

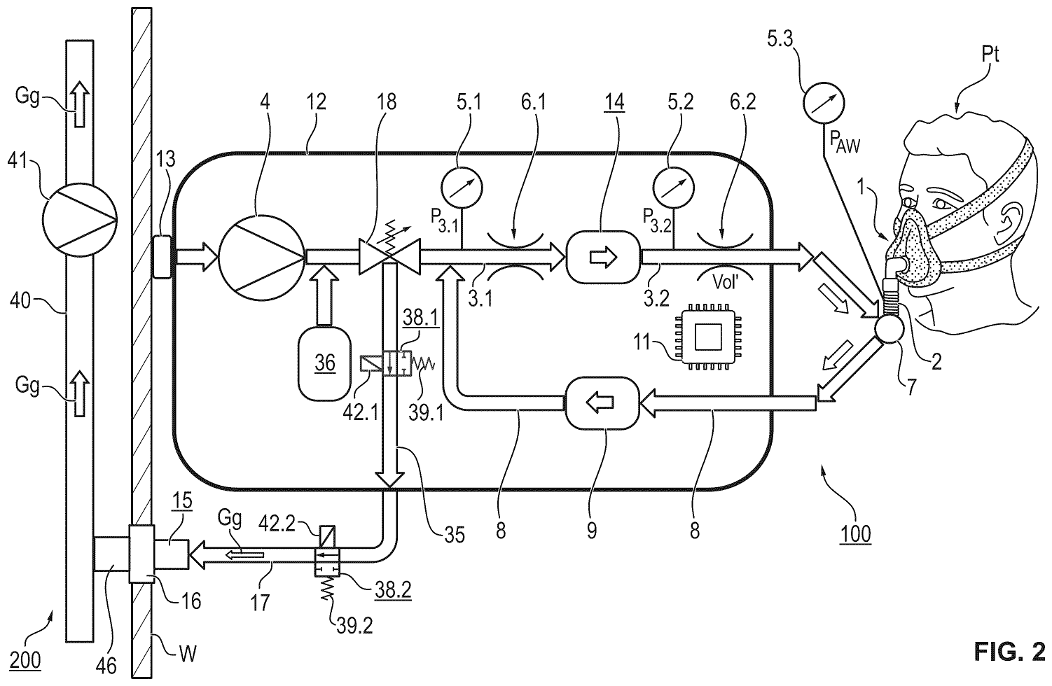

Referring to the drawings, FIG. 1 and FIG. 2 schematically show a preferred implementation of the invention. Identical reference signs have the same meanings.

A patient Pt is artificially ventilated. A patient-side coupling unit is attached to and/or in the body of the patient Pt, in the embodiment example a breathing mask 1 on his/her face. An inspiratory fluid guide unit, for example a tube, comprises two segments 3.1 and 3.2 described below and connects a schematically shown ventilator 12 to a Y-piece 7. The patient-side coupling unit 1 is in a fluid connection with the Y-piece 7 via a fluid guide unit 2.

In the embodiment example, the patient Pt is anaesthetized or at least sedated with the aid of an anesthetic. The ventilator 12 comprises a schematically shown supply connection 13 for breathing air and oxygen and optionally for compressed air and/or for a carrier gas for anesthetics. The supply connection 13 is recessed (inserted) into a wall W. An anesthetic vaporizer 36 feeds anesthetic into a carrier gas and thereby generates a flow of gaseous anesthetic.

The ventilator 12 expels a breathable gas mixture comprising oxygen and at least one anesthetic. Preferably, the ventilator 12 performs a sequence of ventilation strokes and expels in each ventilation stroke a respective quantity of the gas mixture. The expelled gas mixture flows through the inspiratory fluid guide unit 3.1, 3.2 to the Y-piece 7 and through the fluid guide unit 2 and is inhaled by the patient Pt with the aid of the patient-side coupling unit 1.

A fluid conveying unit, for example a blower 4 or a fan or a pump or a piston-cylinder unit, generates a volume flow (volume rate of flow, volume stream), for example a constant volume flow over time, as well as a pressure, for example a constant pressure over time. The constant pressure over time is between 10 mbar and 100 mbar, for example.

A first pressure sensor 5.1 measures the actual pressure P_{3.1} in the first segment 3.1. An optional second pressure sensor 5.2 measures the actual pressure P_{3.2} in the second segment 3.2. A third pressure sensor 5.3 measures the pressure in the airway (pressure in airway, P_{AW}), preferably at a measuring position close to the patient-side coupling unit 1. A first volume flow sensor 6.1 measures the actual volume flow through the first segment 3.1. A second volume flow sensor 6.2 measures the actual volume flow Vol′ through the second segment 3.2.

A signal-processing controller 11 receives a signal from each of the sensors 5.1, 5.2, 5.3 and 6.1, 6.2 and controls a valve arrangement 14 with at least one valve, optionally at least two valves arranged in parallel. The valve arrangement 14 is located between the first segment 3.1 and the second segment 3.2. The controller 11 performs closed-loop control with the control objective (gain) that the actual time course of the volume flow Vol′ through the second segment 3.2 and/or the pressure P_{3.2} in the second segment 3.2 and/or the airway pressure P_{A}W follows a predetermined time course.

Note: The formulation that a sensor is able to measure a physical variable is used repeatedly. This formulation means that the sensor is capable of directly measuring the physical variable or at least one other variable that correlates with the variable to be measured. The or another measured variable or the combination of the measured other variables together are therefore an indicator for the physical variable to be measured. For example, a pressure difference is measured, and the pressure difference is an indicator for a desired volume flow. The measurement provides at least one value for the physical variable sought.

An expiratory fluid guide unit 8, for example another tube, leads from the Y-piece 7 back to the ventilator 12. The gas that the patient Pt has exhaled flows through the expiratory fluid guide unit 8. An end-expiratory valve 9 is preferably arranged in the expiratory fluid guide unit 8, which ensures that a minimum pressure is maintained in the lungs of the patient Pt.

As a rule, the gas exhaled by the patient Pt contains anesthetic. This anesthetic should not enter the environment. Therefore, a ventilation circuit is implemented between the ventilator 12 and the patient-side coupling unit 1 by means of the expiratory fluid guide unit 8. Thanks to the ventilation circuit, the gas exhaled by the patient Pt is fed back into the flow of breathable gas mixture generated by the fluid conveying unit 4.

An excess gas mixture Gg must at least occasionally be diverted (branched-off) from this ventilation circuit. By way of example, a pressure relief valve 18 is shown. The pressure relief valve 18 opens if the pressure in the ventilation circuit is above an upper pressure threshold specified by its design and implementation. A stationary suction arrangement 200 sucks excess gas mixture Gg out of the ventilation circuit. The suction arrangement 200 is arranged behind the wall W. The extracted gas mixture Gg enters a stationary fluid guide network 40 of the hospital. The fluid guide network 40 preferably receives gas mixtures from various ventilators and directs it into an anesthetic conditioner or into the environment.

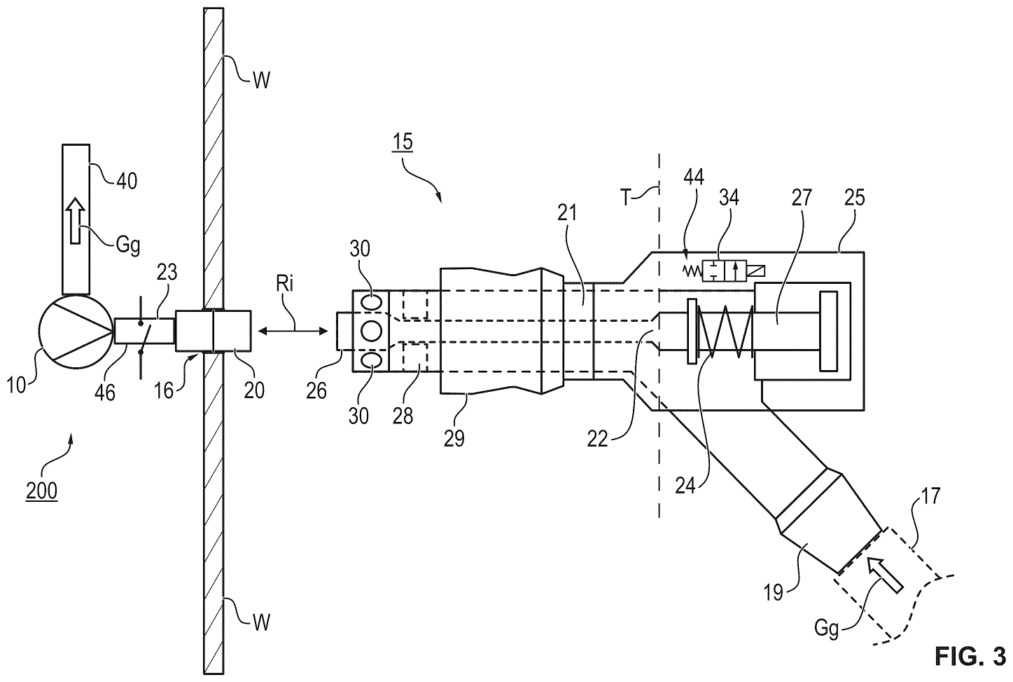

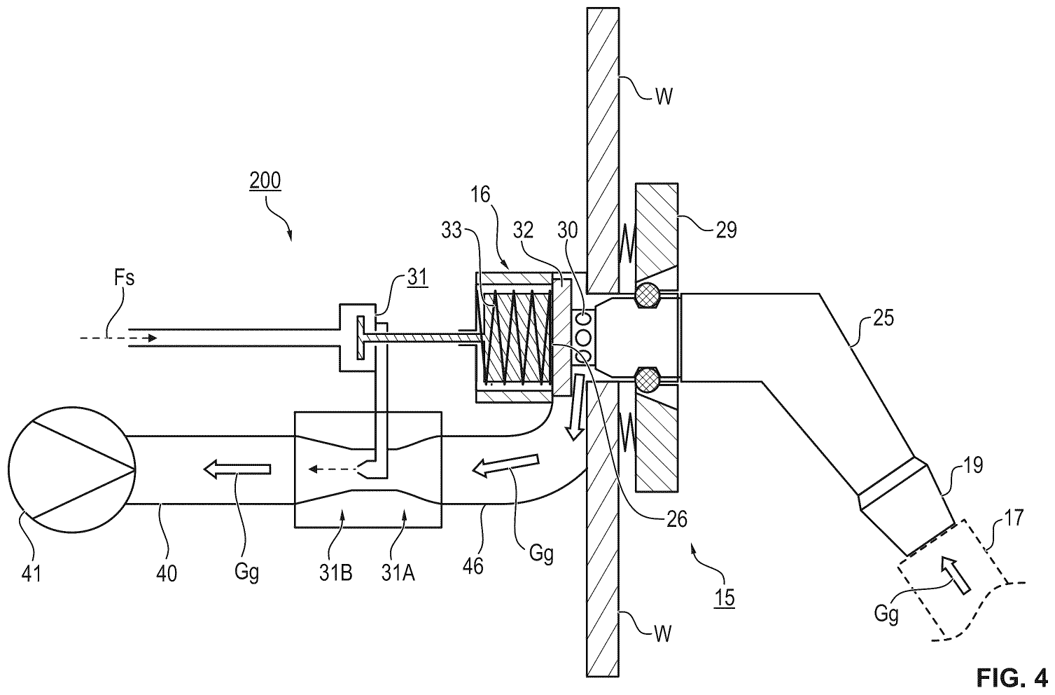

A fluid guide unit 35 in the ventilator 12 leads from the ventilation circuit or from the pressure relief valve 18 to a connection at the housing of the ventilator 12. A fluid guide unit 17, for example a hose, is connected to this connection and guides the branched gas mixture Gg from the ventilator 12 to the wall W. A plug 15 is attached to the free end of the fluid guide unit 17. The fluid guide unit 17 can be connected to a hose connection 19 of the plug 15, see FIG. 3 and FIG. 4. The plug 15 can be plugged into a socket 16 in the wall W and pulled out of the socket 16 again. A lock (latch) 29, for example a snap lock, holds the inserted plug 15 in the socket 16, see FIG. 4. If the plug 15 is inserted into the socket 16, a fluid connection between the ventilator 12 and the stationary fluid guide network 40 is established or can be established. This fluid connection passes through the fluid guide unit 17, the plug 15, and the socket 16. The suction arrangement 200 conveys a gas through the fluid guide unit 17 into the hospital fluid guide network 40.

In the embodiment example, the excess gas mixture Gg is the fluid to be carried away. The pressure relief valve 18 in the ventilator 12 belongs to the source for the fluid Gg, and the fluid guide network 40 acts as the sink. The fluid guide units 35 and 17 together belong to the fluid connection between the source and the sink.

A directional (control) valve 20 or another closure is arranged in the socket 16, see FIG. 3. If the plug 15 is not inserted into the socket 16, the socket 16 is closed and no gas is sucked in through the socket 16 and no gas can escape. If the plug 15 is inserted into the socket 16, the directional (control) valve 20 is opened.

The suction arrangement 200 is part of a scavenging system for the gas mixture. FIG. 1 shows a first embodiment of the scavenging system, FIG. 2 a second embodiment.

In the embodiment shown in FIG. 1, the suction arrangement 200 comprises a fluid conveying unit 10, wherein the fluid conveying unit 10 is configured as a pump, for example. The fluid conveying unit 10 can be switched on and off by a corresponding control. A line 46, e.g. a tube, connects the socket 16 to the fluid conveying unit 10. In the embodiment according to FIG. 2, the suction arrangement 200 comprises a vacuum source 41, which is preferably permanently switched on and preferably has its own power supply. The vacuum source 41 is preferably capable of simultaneously sucking (aspirating) a gas mixture from different ventilators. The line 46 leads to the fluid guide network 40.

In the embodiment shown in FIG. 1, the fluid conveying unit 10 is switched off if the plug 15 is not inserted into the socket 16. The inserted plug 15 actuates a schematically shown contact switch 23, see FIG. 3. The actuated contact switch 23 switches on the fluid conveying unit 10. In the embodiment shown in FIG. 2, the vacuum source 41 is also switched on if the plug 15 is not inserted and the socket 16 is therefore closed.

FIG. 3 and FIG. 4 illustrate two implementations of the plug 15. In FIG. 3, the plug 15 is shown to the right of the dashed vertical dividing line T in a schematic cross-sectional view, and to the left of the dividing line T, the internal contours are shown in dashed line. FIG. 4 shows a schematic cross-sectional view.

The plug 15 has a housing 25. A plurality of openings 30 are inserted into the housing 25. If the plug 15 is inserted into the socket 16, the fluid connection leads from the source 12, 17 through the openings 30, the socket 16, and the line 46 to the sink 40.

A cavity 21 is arranged inside the plug 15, see FIG. 3. A plunger 22 with a tip 26 is movably mounted in this cavity 21 in such a way that the plunger 22 can be moved linearly forwards and backwards in the two opposite directions Ri relative to the rest of the plug 15. A guide 28 guides the plunger 22 during its linear movement in the cavity 21. If the tip 26 of the plunger 22 has moved sufficiently far out of the cavity 21 and through the socket 16 towards the fluid conveying unit 10, the tip 26 protrudes beyond the housing 25. If the plug 15 is inserted and the tip 26 protrudes, the following steps are triggered: the directional control valve 20 is opened, the contact switch 23 is actuated, and the fluid conveying unit 10 is switched on. The plunger 22 acts as the actuating element in the sense of an advantageous embodiment.

A compression spring 24 is supported on the housing 25 and strives to push the tip 26 of the plunger 22 out of the cavity 21 so that the tip 26 protrudes over the front of the housing 25. The compression spring 24 can in particular be a mechanical or pneumatic compression spring. It is possible that several compression springs are arranged in parallel. The compression spring 24 is preferably configured as a purely mechanical component and requires neither a supply of electrical energy nor pneumatic or hydraulic fluid in order to move and hold the plunger 22. The compression spring 24 acts as the resetting element of this embodiment.

An actuator 27 in the plug 15 can be switched on and off by a corresponding control. The actuator 27 can in particular comprise an electric actuator or another electric motor or a piston-cylinder unit or operate electrically, pneumatically or hydraulically in some other way.

In one embodiment, the actuator 27 comprises a hydraulic or pneumatic actuator and an electrically controllable valve, for example a switching valve 34. In a rest state, the switching valve 34 prevents a pneumatic or hydraulic fluid from reaching the actuator. The actuator 27 is then switched off. In response to an electrical activation, the switching valve 34 opens, fluid flows to the actuator of the final control element 27, and the actuator 27 is switched on. Preferably, the resetting force of a spring element 44 holds the switching valve 34 in the closed rest state.

In another embodiment, the actuator 27 comprises an electric motor and a reduction gear, for example a threaded spindle or a toothed rack. The actuator 27 is then configured in such a way that the compression spring 24 is able to move the plunger 22 if the electric motor is switched off and the reduction gear does not inhibit this movement. If the electric motor is switched on, the plunger 22 is pulled into the housing 25 against the resetting force of the compression spring 24.

In both embodiments, the activated actuator 27 is therefore able to pull the plunger 22 completely into the cavity 21 against the resetting force of the compression spring 24, so that the tip 26 no longer protrudes beyond the housing 25. In the embodiment example, the actuator 27 is unilaterally effective, i.e. it can only pull the plunger 22 into the housing 25, but not move it in the opposite direction. If the actuator 27 is switched off, the compression spring 24 displaces (pushes) the plunger 22 out of the housing 25 as just described and holds it.

FIG. 4 shows an embodiment of the suction arrangement 200. In the implementation shown, this embodiment is combined with the second embodiment according to FIG. 2, i.e. with a permanently switched-on vacuum source 41. The embodiment according to FIG. 4 can also be combined with the first embodiment, i.e. with a fluid conveying unit 10 which can be switched on and off.

A pressurized gas valve 31 (a valve for controlling flow from a pressurized gas source) has a suction side 31A and a discharge side 31B, see FIG. 4. The suction side 31A is in a fluid connection with the inserted plug 15, the discharge side 31B is in a fluid connection with the stationary fluid guide network 40. A compression spring 33 strives to close a pressure plate 32. In the embodiment shown in FIG. 4, the compression spring 33 strives to move the pressure plate 32 to the right and thereby close the fluid connection. The compression spring 33 and the pressure plate 32 together form a non-return valve. If the plug 15 is plugged into the socket 16, the tip 26 opens the non-return valve 32, 33. The tip 26 displaces the pressure plate 32 against the spring force of the compression spring 33, namely to the left in the illustration according to FIG. 4. If the non-return valve 32, 33 is open, a fluid flow Fs generates a negative pressure between the intake side 31A and the output side 31B due to the Venturi effect, and the generated negative pressure sucks excess gas mixture Gg out of the inserted plug 15.

The second embodiment shown in FIG. 2 is described below. In this second embodiment, the vacuum source 41 is permanently switched on. The fluid guide units 35 and 17 establish a fluid connection from the ventilation circuit in the ventilator 12 to the socket 16. At least one switching valve is arranged in this fluid connection. This switching valve can either release or block the fluid connection. By way of example, FIG. 2 shows a first switching valve 38.1 in the ventilator 12, namely in the fluid guide unit 35, and a second switching valve 38.2 in the fluid guide unit 17. The or a switching valve can be arranged at one of the positions shown or also in the plug 15. Preferably, a single switching valve is present.

The or each switching valve 38.1, 38.2 comprises a respective mechanical resetting element 39.1, 39.2, for example a mechanical or pneumatic spring, and an actuator 42.1, 42.2. The resetting element 39.1, 39.2 strives to transfer the switching valve 38.1, 38.2 to a releasing position (conveying switching position) and to hold it in the releasing position. If the or each switching valve 38.1, 38.2 is in the releasing position, a fluid connection between the ventilation circuit and the fluid guide network 40 is released, this fluid connection passing through the fluid guide unit 35, 17 and through the plug 15 and the socket 16.

Furthermore, the or each switching valve 38.1, 38.2 each comprises an actuator 42.1, 42.2. The actuator 42.1, 42.2 can be activated externally by a corresponding control. The activated actuator 42.1, 42.2 moves the switching valve 38.1, 38.2 into a blocking position (rest switching position) against the resetting force of the resetting element 39.1, 39.2. If the or at least one switching valve 38.1, 38.2 is in the blocking position, the fluid connection described above is interrupted and the vacuum source 41 is unable to draw in any gas mixture Gg from the ventilation circuit in the ventilator 12.

Both in the first embodiment according to FIG. 1 and FIG. 3 and in the embodiment according to FIG. 2, the following states of the scavenging system are possible:
State 1: The plug 15 is not plugged into the socket 16. Then the directional control valve 20 (FIG. 3) or the non-return valve 32, 33 (FIG. 4) closes the socket 16, and in the first embodiment the fluid conveying unit 10 is switched off (FIG. 3) or the pressurized gas valve 31 is closed (FIG. 4).
State 2: The plug 15 is plugged into the socket 16. In the first embodiment (FIG. 1 and FIG. 3), the actuator 27 is switched off. In the embodiment shown in FIG. 3, the compression spring 24 presses the tip 26 against the contact switch 23 and the fluid conveying unit 10 is switched on. In the embodiment shown in FIG. 4, the tip 26 opens the non-return valve 32, 33 and the pressurized gas valve 31. In the second embodiment (FIG. 2), the switching valve 38.1, 38.2 is in the releasing position, and the resetting element 39.1, 39.2 holds it in the releasing position.
In state 2, in both embodiments a gas mixture Gg is drawn through the fluid guide unit 17 and the plug 15 into the fluid guide network 40.
State 3: The plug 15 is plugged into the socket 16. In the first configuration, the actuator 27 is switched on and pulls the plunger 22 back against the force of the compression spring 24. The tip 26 no longer touches the contact switch 23 or no longer opens the pressurized gas valve 31, and the fluid conveying unit 10 is switched off (FIG. 3) or the pressurized gas valve 31 is closed (FIG. 4). In the second embodiment, the actuator 42.1, 42.2 holds the switching valve 38.1, 38.2 in the blocking position.

Excess gas mixture Gg is not extracted in state 3 in either embodiment.

In state 1, no fluid can pass through the socket 16 into the fluid guide network 40. In the first embodiment, the fluid conveying unit 10 is switched off.

In state 2, the suction arrangement 200 in both embodiments sucks the gas mixture Gg through the plug 15 in the socket 16 into the fluid guide network 40. In state 2, this prevents that too much excess gas mixture Gg accumulates in the ventilator 12. This is a safe state.

In state 3, the plug 15 is plugged into the socket 16. However, in the first embodiment, the fluid conveying unit 10 is switched off, and in the second embodiment, the or each switching valve 38.1, 38.2 is in the blocking position. This saves electrical energy. In both embodiments, the quantity of the gas mixture Gg that enters the fluid guide network 40 is reduced compared to an intake (suction).

State 3 may occur if the plug 15 is plugged into the socket 16, but the ventilator 12 is not currently performing artificial ventilation and is also not ready to perform artificial ventilation. For example, after using the ventilator 12, the step has not yet been performed or has been forgotten that the plug 15 is removed from the socket 16, or the plug 15 has been inserted but artificial ventilation has not yet started. In this case, it makes sense for the suction arrangement 200 to be switched off or at least not to suck in any gas. The actuator 27 produces this desired effect.

The undesired event can occur if an electrical or hydraulic or pneumatic supply to the actuator 27 is interrupted or the actuator 27 fails. In this case, excess gas mixture Gg must continue to be extracted and the suction arrangement 200 must continue to operate and must not be switched off.

If the actuator 27 is switched off or has failed, the compression spring 24 pushes the plunger 22 out of the housing 25 and the suction arrangement 200 is transferred to the safe state 2. This transfer does not require any intervention by a user and additionally does not require any electrical or pneumatic or hydraulic supply to a component. Preferably, this transfer is carried out regardless of whether the ventilator 12 is ready for operation or not.

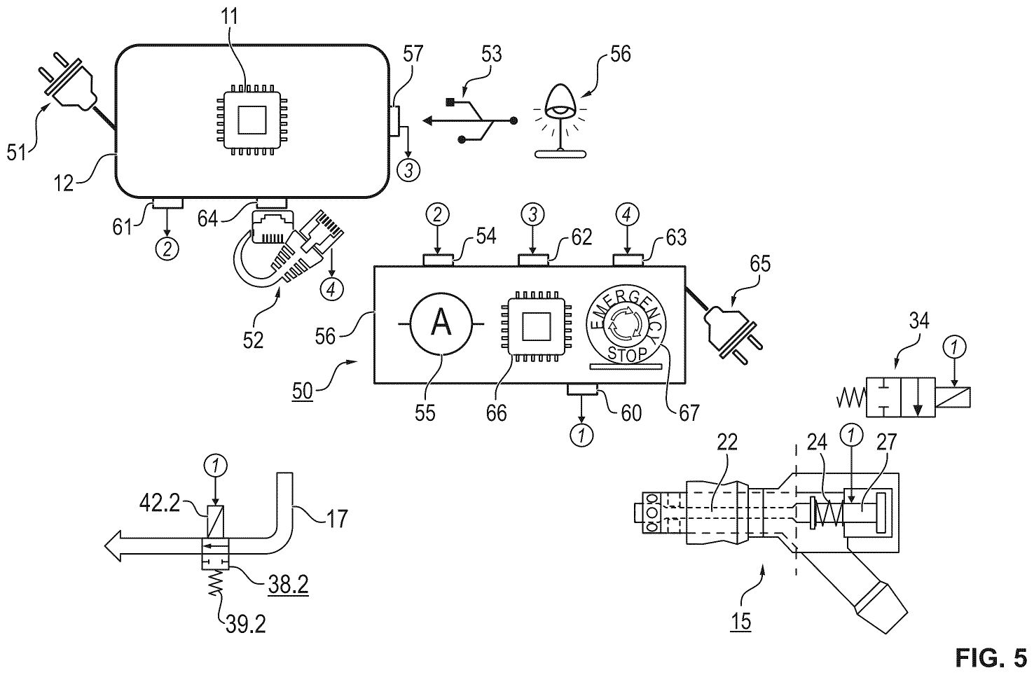

FIG. 5 schematically shows a power plug 51 of the ventilator 12 and a signal-processing control unit 50 with its own data-processing processor 66. This control unit 50 is in a data connection with the actuator 27 of the plug 15 or with the actuator 42.1, 42.2 of the switching valve 38.1, 38.2 and is able to switch the actuator 27, 42.1, 42.2 on and/or off. As an example, it is shown that the control unit 50 can cause via a data connection the switching valve 34 for the actuator 27 or the actuator 42.1, 42.2 of the switching valve 38.1, 38.2 to open or to close. The control unit 50 comprises an output 60 in the form of a communication port, via which the data connection with the switching valve 34 or the actuator 42.1, 42.2 is or can be established. Optionally, the control unit 50 supplies an actuator for the switching valve 34 or the actuator 42.1, 42.2 with electrical energy.

In the implementation just described, the control unit 50 is able to control the actuator 27, 42.1, 42.2 via a data connection. It is also possible that a fluid connection can be established between the control unit 50 and the actuator 27, 42.1, 42.2 and that the control unit 50 supplies the actuator 27, 42.1, 42.2 with a hydraulic or pneumatic fluid via this fluid connection and thereby activates it.

Preferably, the control unit 50 also comprises its own housing 56 and is preferably arranged outside the ventilator 12. It is possible that the ventilator 12 is configured to supply the control unit 50 with electrical energy. Preferably, however, the control unit 50 has its own power plug 65, so that the control unit 50 can be supplied with electrical energy independently of the ventilator 12. The control unit 50 can additionally or instead have its own power supply unit (not shown) in order to be independent of a stationary power supply network. It is also possible that the power plug 51 of the ventilator 12 can be plugged into a corresponding socket (not shown) in the housing 56 of the control unit 50 or, conversely, that the power plug 56 can be plugged into a corresponding socket (not shown) in the housing of the ventilator 12.

The implementation that the control unit 50 is a device with its own housing 56 and preferably with its own power supply makes it easier to retrofit an existing scavenging system with a control unit 50 according to the invention without having to make significant changes to the ventilator 12.

The control unit 50 is preferably configured as follows: If the control unit 50 is not supplied with electrical voltage or is switched off or has failed, the actuator 27 is switched off. This establishes a safe state.

The ventilator 12 can be connected to a stationary power supply network using a standard power plug 51. Via a data output 61 of the ventilator 12 and an input 54 of the control unit 50, the control unit 50 can determine what electrical current the ventilator 12 is drawing with the aid of the power plug 51. By way of example, a current (amperage) sensor 55 is shown, which sensor is connected to the input 54. This implementation enables the control unit 50 to automatically decide whether the ventilator 12 is connected to the power supply network and switched on.

In the embodiment example, the ventilator 12 is able to supply an illumination (lighting) unit 56 with electrical voltage. The illumination unit 56 is able to illuminate a writing pad or display surface of the ventilator 12. The illumination unit 56 is connected to a USB plug 53, which can be plugged into a USB socket 57. The illumination unit 56 is supplied with electrical voltage via this USB connection 53, 57. The control unit 50 is able to monitor the USB socket 57 with the aid of a data input 62. The control unit 50 is able to automatically decide whether an external device 56 is supplied with electrical voltage via the USB plug 57 and/or whether an electrical current is flowing. In addition to or instead of the USB socket 57, the control unit 50 can also monitor a voltage output of the ventilator 12 for standard AC voltage or DC voltage. If the ventilator 12 supplies an electrical load via this voltage output, the ventilator 12 is generally switched on and is performing artificial ventilation or is at least ready to perform artificial ventilation.

A data connection 52 can be established between the ventilator 12 and the control unit 50, for example using an Ethernet cable or a wired data network, e.g. in accordance with the “Service-Oriented Device Connectivity” (SDC) standard, or wirelessly via radio waves. This data connection 52 leads from a data output 64 of the ventilator 12 to a data input 63 of the control unit 50. In one embodiment, a data cable for the data connection 52 is connected to a cable for the power plug 51 of the ventilator 12. This embodiment reduces the risk of the data connection 52 being unintentionally interrupted.

Via this data connection 52, the control unit 50 is able to detect a current operating state of the ventilator 12. Possible operating states are, for example:
The ventilator 12 is in a ventilation mode and is currently performing artificial ventilation of the patient Pt or is at least ready to perform artificial ventilation.
The ventilator 12 is switched on and is in a standby mode.
The ventilator 12 is switched off.

In one embodiment, the control unit 50 is additionally able to determine an operating parameter of the ventilator 12, for example the generated volume flow through the first segment 3.1, wherein the first volume flow sensor 6.1 has measured this volume flow, or the generated airway pressure P_{AW}. If the volume flow or the airway pressure P_{AW} remains below a predetermined upper threshold for a sufficiently long period of time, artificial ventilation has not yet started or has already finished.

The control unit 50 is able to detect the following event by means of a signal that is transmitted via the data link 52: The ventilator 12 has finished artificial ventilation of the patient Pt. In this case, the ventilator 12 is first switched from a ventilation mode to a rest (idle) state or sleep mode and then switched off. It is also possible that a message is explicitly transmitted via the data connection 52 described above, which message specifies a switch-off process for the ventilator 12. The control unit 50 receives and processes this message.

In addition, the control unit 50 is able to detect the event that the data connection 52 is suddenly interrupted, for example because the Ethernet connection was accidentally disconnected, e.g. as the cable was pulled. In this case, a sleep mode does not occur beforehand. It is also possible that the data connection 52 is continuously monitored to see whether it is still being maintained (Alive Protocol).

A state 2 and a state 3 were described above. In state 2, the actuator 27 is switched off, the suction arrangement 200 is switched on, and a fluid connection is established between the ventilator 12 and the fluid guide network 40, which passes through the fluid guide unit 17 and the plug 15. Excess gas mixture Gg is extracted, e.g. sucked. In state 3, the actuator 27, 42.1, 42.2 is switched on, and the switched-on actuator 27, 42.1, 42.2 causes the suction arrangement 200 to be switched off or the fluid connection to be interrupted and therefore no excess gas mixture Gg is sucked off and thus energy is saved.

The following description refers to the situation in which the plug 15 is plugged into the socket 16. Ideally, if the plug 15 is plugged in, state 2 occurs while the ventilator 12 is performing artificial ventilation or is at least ready for operation, and state 3 otherwise occurs. It must surely be ensured that state 2 is established while the ventilator 12 is performing artificial ventilation or is at least ready for operation. If the ventilator 12 is not performing artificial ventilation and is also not ready for operation, state 3 should be established. However, if state 2 is established instead, electrical and/or pneumatic energy is consumed, but no safety-critical situation occurs. This safe state is achieved on the one hand by the following function: The resetting element 24, 39.1, 39.2 ensures that state 2 is established if the actuator 27, 38.1, 38.2 or the control unit 50 has failed or is switched off. This function ensures that state 2 is established at least during artificial ventilation, but only relatively rarely, ideally not at all, if the ventilator 12 is not performing artificial ventilation.

The following describes how a safety-critical situation can be avoided with the aid of the control unit 50 while still saving electrical energy. The control unit 50 applies a predetermined computer-evaluable decision logic. The decision logic is a logical combination of the occurrence or non-occurrence of several predetermined possible events and determines whether the actuator 27, 42.1, 42.2 is switched on (state 3) or off (state 2).

The control unit 50 is able to automatically decide for each possible event that is considered in the decision logic whether this possible event has actually occurred or not. In the example embodiment, the following are possible events:
The ventilator 12 performs artificial ventilation or is being prepared (ready) to perform artificial ventilation. The ventilator 12 therefore consumes current, wherein the amperage is above a predefined lower amperage threshold. This is detected using the current sensor 55, for example. State 2 must then be present.
The ventilator 12 has terminated artificial ventilation. The control unit 50 uses the data connection 52 to determine that the ventilator 12 is being switched from ventilation mode to sleep mode and then switched off. State 3 can now be established.
Artificial ventilation has not yet started or has finished. The control unit 50 detects that the volume flow through the first segment 3.1 or the airway pressure P_{A}w is less than a predefined upper threshold for a sufficiently long time period. State 3 can then also be established.
A message is transmitted via the data connection 52 described above. This message contains the information that the ventilator 12 is in a sleep mode (a standby mode). State 3 can then also be established.
An electrical consumer (load) is connected to the ventilator 12. For example, the light source 56 is connected to the USB socket 57 using the USB plug 53. State 2 should then be present, unless the control unit 50 has positively detected the event that the ventilator 12 is in a sleep mode.
The data connection 52 is interrupted without a sleep mode having been detected beforehand and without a message indicating an imminent switch-off having been transmitted. State 2 should then be present.

In one embodiment, the control unit 50 additionally comprises an emergency stop switch 67. If a user actuates the emergency stop switch 67, the control unit 50 is switched off, thereby also switching off the actuator 27, and state 2 is established.

While specific embodiments of the invention have been shown and described in detail to illustrate the application of the principles of the invention, it will be understood that the invention may be embodied otherwise without departing from such principles.

List of reference symbols

| | |
|---|---|
| 1 | Patient-side coupling unit in the form of a breathing mask, connected to |
| | the fluid guide unit 2 and to the patient Pt |
| 2 | Fluid guide unit, connects the Y-piece 7 with the patient-side coupling |
| | unit 1 |
| 3.1 | First segment of the inspiratory fluid guide unit, leads from the fluid |
| | conveying unit 4 to the valve arrangement 14 |
| 3.2 | Second segment of the inspiratory fluid guide unit, leads from the valve |
| | arrangement 14 to the Y-piece 7 |
| 4 | Fluid conveying unit in the form of a blower, expels a gas mixture into |
| | the first segment 3.1, is connected to the supply connection 13 |
| 5.1 | First pressure sensor, measures the actual pressure P_{3.1} in the first segment |
| | 3.1, wherein the pressure P_{3.1} is usually generated by the fluid conveying |
| | unit 4 |
| 5.2 | Second pressure sensor, measures the actual pressure P_{3.2} in the second |
| | segment 3.2 |
| 5.3 | Third pressure sensor, measures the actual airway pressure P_{AW} |
| 6.1 | First volume flow sensor, measures the actual volume flow through the |
| | first segment 3.1 |
| 6.2 | Second volume flow sensor, measures the actual volume flow Vol' |
| | through the second segment 3.2 |
| 7 | Y-piece, connects the fluid guide units 3.2 and 8 on one side with the |
| | fluid guide unit 2 on the other side |
| 8 | Expiratory fluid guide unit, leads from the Y-piece 7 back to the |
| | ventilator 12 |
| 9 | End-expiratory valve in the expiratory fluid guide unit 8 |
| 10 | Fluid conveying unit of the suction arrangement 200, can be switched on |
| | and off, is able to suck the gas mixture Gg into the fluid guide network 40 |
| 11 | Signal-processing controller, receives and processes setting parameter |
| | signals from the sensors 5.1, 5.2 and 6.1, 6.2, controls the valve |
| | arrangement 14 and, in the first embodiment, the switching valve 34 and, |
| | in the second embodiment, the switching valves 38.1, 38.2 |
| 12 | Ventilator, comprises the fluid conveying unit 4, the controller 11, the |
| | valve arrangement 14, the actuator arrangement 20, the sensors 5.1, 5.2 |
| | and 6.1, 6.2 and the supply connection 13 |
| 13 | Supply connection of the ventilator 12, connected to the fluid supply unit |
| | 4 |
| 14 | Valve arrangement, arranged between segments 3.1 and 3.2 |
| 15 | Plug at the free end of the fluid guide unit 17, comprises in one |
| | embodiment the housing 25, the plunger 22, the compression spring 24, |
| | the actuator 27, the lock 29 and the switching valve 34 |
| 16 | Socket in the wall W, accepts the plug 15 |
| 17 | Fluid guide unit, leads from the fluid guide unit 35 to the plug 15, |
| | connected or connectable to the hose connection 19, is optionally released |
| | or closed by the switching valve 38.2 in one embodiment |
| 18 | Pressure relief valve in the ventilation circuit |
| 19 | Hose connection on plug 15, can be connected to the fluid guide unit 17 |
| 20 | Shut-off valve in the socket 16, can be opened by the plug 15 |
| 21 | Cavity inside the plug 15, accommodates the plunger 22 |
| 22 | Plunger in the cavity 21, can be moved linearly in the two opposite |
| | directions Ri, has the tip 26 |
| 23 | Contact switch in the socket 16 |
| 24 | Compression spring in the plug 15, supported by the housing 25, strives to |
| | push the tip 26 of the plunger 22 out of the cavity 21 |
| 25 | Housing of the plug 15 |
| 26 | Tip of the plunger 22, is able to open the shut-off valve 20 and the |
| | pressurized gas valve 31 |
| 27 | Actuator in the plug 15, strives to pull the plunger 22 into the cavity 21 |
| | against the force of the compression spring 24, is switched on and off by |
| | the control unit 50 |
| 28 | Guide in the cavity 21 for the plunger 22 |
| 29 | Lock (latch) for the plug 15 |
| 30 | Openings in the housing 25 |
| 31 | Pressurized gas valve, has the suction side 31A and the discharge side |
| | 31B |
| 31A | Suction side of the pressurized gas valve 31 |
| 31B | Output side of the pressurized gas valve 31 |
| 32 | Contact switch in the form of a pressure plate |
| 33 | Compression spring for the pressure plate 32 |
| 34 | Controllable switching valve which releases or interrupts the flow of a |
| | fluid to the actuator 27 |
| 35 | Fluid guide unit in the ventilator 12, connects the ventilation circuit with |
| | the fluid guide unit 17 |
| 36 | Anesthetic vaporizer |
| 38.1, 38.2 | Controllable switching valve, which releases or interrupts a fluid |
| | connection between the ventilator 12 and the socket 16, comprises the |
| | resetting element 39.1, 39.2 and the actuator 42.1, 42.2 |
| 39.1, 39.2 | Mechanical resetting element for the switching valve 38.1, 38.2 |
| 40 | Stationary fluid guide network of the hospital, absorbs excess gas mixture |
| | Gg extracted by suction |
| 41 | Vacuum source, is permanently switched on, sucks the gas mixture Gg |
| | into the fluid guide network 40 |
| 42.1, 42.2 | Controllable actuator of the switching valve 38.1, 38.2 |
| 44 | Spring element, keeps the switching valve 34 in the closed position |
| 46 | Line from the socket to the fluid guide network 40 |
| 50 | Signal-processing control unit, comprises the housing 56, 10 processor |
| | 66, the power plug 65, the emergency stop switch 67, the data inputs 54, |
| | 62, 63 and the data output 60, switches the actuator 27 on and off in the |
| | first embodiment and the actuators 42.1, 42.2 in the second embodiment |
| 51 | Power plug of the ventilator 12 |
| 52 | Data connection between the ventilator 12 and the control unit 50 is |
| | established via the data output 54 and the data input 63 |
| 53 | USB plug of the illumination unit 56, can be plugged into the USB socket |
| | 57 |
| 54 | Data input of the control unit 50, connected to the current sensor 55 |
| 55 | Current sensor, connected to input 54 |
| 56 | Illumination unit for a writing pad of the ventilator 12, connected to the |
| | USB plug 53 |
| 57 | USB socket into which the USB plug 53 can be inserted |
| 58 | Housing of the control unit 50 |
| 60 | Data output in the form of a communication port via which the data |
| | connection is established with the switching valve 34 or the actuator 42.1, |
| | 42.2 |
| 61 | Data output of the ventilator 12, used for monitoring the USB socket 57 |
| 62 | Data input of the control unit 50, is used for monitoring the USB socket |
| | 57 |
| 63 | Data input of the control unit 50, is used for the data connection 52 |
| 64 | Data output of the ventilator 12, is used for the data connection 52 |
| 65 | Power plug of the control unit 50 |
| 66 | Processor of the control unit 50 |
| 67 | Emergency stop switch of the control unit 50 |
| 100 | Ventilation arrangement, comprising the ventilator 12, the fluid guide |
| | units 2, 3.1, 3.2, 8, the Y-piece 7 and the patient-side coupling unit 1 |
| 200 | Suction arrangement in the wall W, comprises in one embodiment the |
| | fluid conveying unit 10 and in another embodiment the pressurized gas |
| | valve 30 |
| Fs | Fluid flow that extracts gas mixture Gg |
| Gg | Excess gas mixture, is branched off from the ventilation circuit and |
| | conveyed through the plug 15 into the fluid guide network 40, acts as the |
| | fluid |
| P_{AW} | Pressure in the second segment 3.2, measured by pressure sensor 5.2 |
| Pt | Patient, is artificially ventilated, connected to the patient-side coupling |
| | unit 1 |
| Ri | Opposite directions in which the plunger 22 can be moved |
| T | Dividing line |
| Vol' | Volume flow through the second segment 3.2, measured by the volume |
| | flow sensor 6.2 |
| W | Wall, behind which the suction arrangement 200 and the fluid guide |
| | network 40 are located, the socket 16 has |

What is claimed is:

1. A scavenging system for a fluid, the scavenging system comprising:
a switching element;
a resetting element configured to reset the switching element;
an actuator that is configured to be switched on and switched off;
a signal-processing control unit;
a socket; and
a plug, which is configured to be inserted into the socket, wherein the plug and socket are configured such that a fluid connection between a fluid source and a fluid sink is or can be established with the plug inserted into the socket; and
a suction arrangement, which is configured to convey fluid from the source to the sink,
wherein the switching element is configured to be switched to a conveying switching position in which the switching element sets the scavenging system to a conveying state and to a rest switching position in which the switching element sets the scavenging system to a rest state,
wherein, in the conveying state, the scavenging system is configured to convey, by using the suction arrangement, the fluid to the sink,
wherein, in the rest state, the scavenging system is configured to interrupt and/or prevent conveying of fluid,
wherein the resetting element is implemented as a mechanical component, has a rest state, and is configured to exert a resetting force if being deflected from the rest state,
wherein the resetting element in the rest state holds the switching element in the conveying switching position and the resetting force strives to switch the switching element into the conveying switching position,
wherein the actuator is configured to switch, if the actuator is switched on, the switching element into the rest switching position against the resetting force of the resetting element, and to enable, if the actuator is switched off, the resetting element to switch the switching element to the conveying switching position,
wherein the signal-processing control unit is configured to monitor the scavenging system and/or the source for at least one event in which no fluid escapes from the source and, if the control unit has positively detected the event or at least one event in which no fluid escapes from the source, to switch the actuator to the actuator switched-on state, and
wherein the scavenging system is configured such that, at least if the signal-processing control unit has not positively detected that no fluid is escaping from the source, the actuator is switched off.

2. A scavenging system according to claim 1, wherein the signal-processing control unit is configured to positively detect the event that the source or a device comprising the source is switched off as an event in which no fluid escapes from the source.

3. A scavenging system according to claim 1,
wherein the signal-processing control unit is configured to detect at least one indication that fluid is escaping from the source and/or the source is ready to expel fluid, and
wherein the signal-processing control unit is configured to cause the actuator to be switched off, or to ensure that the actuator is or will be switched off, upon detection of the indication that fluid is escaping from the source and/or the source is ready to expel fluid to.

4. A scavenging system according to claim 3,
wherein the signal-processing control unit is configured to detect an event that the source receives, consumes, or outputs electrical energy, as an indication that fluid is escaping from the source and/or that the source is operable to expel fluid, and
wherein the detected energy received, consumed, or output by the source is above a predetermined lower threshold.

5. A scavenging system according to claim 1, wherein the signal-processing control unit is configured to detect an indication that a data connection between the source and the signal-processing control unit is interrupted, and with detection of this indication, to cause the actuator to be switched off or to ensure that the actuator is or will be switched off.

6. A scavenging system according to claim 1,
wherein the suction arrangement is configured to be switched on to a switched-on state and switched off to a switched-off state,
wherein the plug comprises a movably mounted actuating element,
wherein the resetting element and the actuator are components of the plug,
wherein the scavenging system is in the conveying state with the suction arrangement switched on and the scavenging system is in the rest state with the suction arrangement switched off,
wherein the switching element is configured to hold the suction arrangement in the switched-on or switched-off state depending on its switching position,
wherein the actuating element is configured to be moved into an actuating position, and
wherein the actuating element in the actuating position is configured to actuate the switching element in such a way that the suction arrangement is or remains in the switched-on state.

7. A scavenging system according to claim 6,
wherein the resetting element being in the rest state holds the actuating element in the actuating position and thereby holds the suction arrangement in the switched-on state, and
wherein the switched-on actuator moves the actuating element out of the actuating position against the resetting force of the resetting element and by this causes the suction arrangement to be switched off to the switched-off state.

8. A scavenging system according to claim 1,
wherein the switching element comprises at least one switching valve,
wherein the switching valve is arranged in the fluid connection leading from the source to the sink,
wherein the switching valve is configured to be switched into a conveying switching position and into a rest switching position,
wherein the switching valve in the conveying switching position releases the fluid connection and the switching valve in the rest switching position interrupts the fluid connection,
wherein the resetting element in the rest state holds the switching valve in the conveying switching position, and
wherein the switched-on actuator is configured to switch the switching valve into the rest switching position against the resetting force of the restoring element.

9. A scavenging system according to claim 1, the source is a component of a device, and the control unit comprises a housing arranged spatially spaced from the device with the source.

10. A system comprising:
a fluid source for a fluid;
a fluid sink for the fluid;
a scavenging system, the scavenging system comprising: a switching element; a resetting element configured to reset the switching element; an actuator configured to be switched on and switched off; a signal-processing control unit; a socket; and a plug, which plug is configured to be inserted into the socket,
wherein the plug and socket are configured such that a fluid connection between the fluid source and the fluid sink is or can be established with the plug inserted into the socket; and a suction arrangement, which is configured to convey fluid from the source to the sink,
wherein the switching element is configured to be switched to a conveying switching position in which the switching element sets the scavenging system to a conveying state and to a rest switching position in which the switching element sets the scavenging system to a rest state,
wherein, in the conveying state, the scavenging system is configured to convey, by using the suction arrangement, the fluid to the sink,
wherein, in the rest state, the scavenging system is configured to interrupt and/or prevent conveying of fluid,
wherein the resetting element is implemented as a mechanical component, has a rest state, and is configured to exert a resetting force if being deflected from the rest state, wherein the resetting element in the rest state holds the switching element in the conveying switching position and the resetting force strives to switch the switching element into the conveying switching position,
wherein the actuator is configured to switch, if the actuator is switched on, the switching element into the rest switching position against the resetting force of the resetting element, and to enable, if the actuator is switched off, the resetting element to switch the switching element to the conveying switching position,
wherein the signal-processing control unit is configured to monitor the scavenging system and/or the source for at least one event in which no fluid escapes from the source and, if the control unit has positively detected the event or at least one event in which no fluid escapes from the source, to switch the actuator to the actuator switched-on state, and
wherein the scavenging system is configured such that, at least if the signal-processing control unit has not positively detected that no fluid is escaping from the source, the actuator is switched off, wherein the fluid connection is interrupted if the plug is not inserted.

11. A system according to claim 10, further comprising a ventilator,
wherein the sink comprises an intake arrangement for the fluid,
wherein the ventilator is configured for artificial ventilation of a patient,
wherein the source is a component of the ventilator,
wherein a patient-side coupling unit is arranged in and/or on the patient's body during artificial ventilation,
wherein a fluid connection between the ventilator and the patient-side coupling unit is established at least temporarily during artificial ventilation, and
wherein the fluid stems from the ventilator and/or the fluid stems from the connection between the ventilator and the patient-side coupling unit and is conveyed to the sink.

12. A system according to claim 11,
wherein, during artificial ventilation, a ventilation circuit is established between the ventilator and the patient-side coupling unit and
wherein the fluid stems from the ventilation circuit, comprises at least one anesthetic and/or medication, and is conveyed to the sink.

13. A process for conveying a fluid from a source to a sink,
wherein the process is performed by using a scavenging system,
wherein the scavenging system comprises a suction arrangement, a switching element, a resetting element for the switching element, a signal-processing control unit, an actuator that can be switched on and off, a plug and a socket,
wherein the resetting element is implemented as a mechanical component and has a rest state,
wherein the process comprises the steps of:
inserting the plug into the socket or having a state with the plug inserted into the socket; and
switching the switching element to a conveying switching position or having the switching element in the conveying switching position, wherein the switching element in the conveying switching position sets the scavenging system to a conveying state,
wherein with the scavenging system in the conveying state, the process further comprises:
establishing a fluid connection between the source and the sink or having such a fluid connection being established;
with the suction arrangement, conveying the fluid through the fluid connection from the source to the sink;
switching off the actuator;
with the resetting element, holding the switching element in the conveying switching position in the rest state;
with the resetting element being deflected from the rest state, exerting a resetting force, wherein the exerted resetting force strives to switch the switching element to the conveying switching position; and
with the signal-processing control unit, monitoring the scavenging system and/or the source for at least one event in which no fluid escapes from the source,
wherein, with the plug inserted, if the signal-processing control unit positively detects the event or at least one event in which no fluid escapes from the source, the process further comprises:
with the signal-processing control unit, switching on the actuator;
with the actuator switched on, switching the switching element into the rest switching position against the resetting force of the resetting element;
with the switching element in the rest switching position causing the scavenging system to be set to a rest state, and
with the scavenging system in the rest state, interrupting and/or preventing the conveyance of fluid through the fluid connection or having such a conveyance being interrupted and/or prevented.

14. A process according to claim 13,
wherein the suction arrangement is configured to be switched on to a switched-on state and switched off to a switched-off state,
wherein the plug comprises a movably mounted actuating element,
wherein the resetting element and the actuator are components of the plug,
wherein the scavenging system is in the conveying state while the suction arrangement is switched on, and the scavenging system is in the rest state while the suction arrangement is switched off,
wherein, while the scavenging system is in the conveying state, the process further comprises:
with the resetting element in the rest state, holding the actuating element in an actuating position,
with the actuating element, in the actuating position, actuating the switching element;
with the actuated switching element, setting the suction arrangement to the switched-on state and/or maintaining the suction arrangement in the switched-on state,
wherein the step that, with the switched-on actuator, the switching element is switched to the rest switching position comprises the step of, with the switched-on actuator, moving the actuating element against the resetting force of the resetting element out of the actuating position and by this causing the suction arrangement to be set to the switched-off state.

15. The process according to claim 13,
wherein the switching element comprises at least one switching valve,
wherein the switching valve is arranged in the fluid connection leading from the source to the sink,
wherein, while the scavenging system is in the conveying state, the process further comprises the steps of:
with the resetting element in the rest state, switching the switching valve to a conveying switching position and/or holding the switching valve in the conveying switching position and
with the switching valve, releasing the fluid connection in the conveying switching position,
wherein the step that the switched-on actuator switches the switching element to the rest switching position comprises the step that the switched-on actuator switches the switching valve to the rest switching position against the resetting force of the resetting element and thereby causes the scavenging system to be set to the rest state.
